(19) United States
(12) Patent Application Publication
(10) Pub. No.: US 2006/0142390 A1
(43) Pub. Date: Jun. 29, 2006

(54) Treatment for severe melancholic depression

(76) Inventors: Mehar Manku, Stirling (GB); Harald Murck, Stirling (GB)

Correspondence Address: JACOBSON HOLMAN PLLC, 400 SEVENTH STREET N.W., SUITE 600, WASHINGTON, DC, 20004, US

(21) Appl. No.: 11/066,329

(22) Filed: Feb. 25, 2005

(30) Foreign Application Priority Data
Dec. 23, 2004 (GB) 0428318.0

(51) Int. Cl. A61K 31/202

(52) U.S. Cl. 514/560

(57) ABSTRACT

Methods for treating severe melancholic depression, comprising administering, to a subject, a preparation comprising EPA, or metabolite(s) thereof.

The present invention relates the treatment of patients suffering from severe melancholic depression and methods for assessing whether depressed patients will benefit from a particular treatment.

Depression is a serious, and complex, disorder affecting millions of people worldwide. In recent years, Selective Serotonin Reuptake Inhibitors (SSRIs) have been used to treat patients suffering from depression. Essential fatty acids have also been recognised as possible treatments for patients suffering from this disorder (WO 00/44361 and WO98/16216). However, to date, both of these treatments have been used as a generic treatment for depression despite it being known that the symptoms, which manifest themselves in patients suffering from depression, vary considerably.

A subset of depression is the more serious disorder of severe melancholic depression. The Hamilton Depression Rating Scale (HDRS) (Hamilton M. A Rating Scale for Depression Defined; J. Neurol Psyciatry 1960; 23: 56-62) provides a means for testing the severity of a patient's depression, however, even with this scale, it is often difficult to prescribe suitable treatments to depressed patients. Even if patients suffering from severe melancholic depression are identified, there is only a limited number of effective treatments available. In the past, SSRIs have been used to treat patients suffering from severe melancholic depression but with only limited success. Therefore, there remains a desire to find alternative, improved ways of treating severely melancholic patients and identifying patients who will benefit from treatment.

The present invention relates to a method for treating severe melancholic depression, comprising administering, to a subject, a preparation comprising EPA.

The present invention also relates to the use of EPA in the manufacture of a medicament for the treatment of severe melancholic depression.

EPA is a highly unsaturated fatty acid which can be derived from the dietary essential fatty acid, α-linolenic acid by a series of three reactions (FIG. 1). EPA is a fatty acid containing 20 carbon atoms and 5 double bonds, all in the cis-configuration. The double bonds are located at the 5, 8, 11, 14 and 17 positions and the full chemical name is therefore all cis (or all z) 5,8,11,14,17-eicosapentaenoic acid (or sometimes icosapentaenoic acid). The abbreviation, which is always used, is EPA.

EPA is one of the highly unsaturated fatty acids, the main types of which are shown in FIG. 2. The reactions which convert alpha-linolenic acid to EPA are slow in humans and only a very small proportion of dietary α-linolenic acid is converted to EPA. EPA is also found in marine micro-organisms and, via the food chain, makes up between 3% and 30% of natural marine oils derived from oily fish and marine mammals. EPA is found linked to many different chemical structures. It can be found in the form of phospholipids, tri, di- and monoglycerides, amides, esters of many different types, salts and other compounds. In each case the EPA moiety can normally be split from the complex molecule to give the free acid form which can then be linked again to other complex molecules.

Conventionally, most studies on the uses of EPA and related fatty acids have used materials partially enriched in EPA but also containing substantial amounts of other fatty acids, especially docosahexaenoic acid (DHA) which is found alongside EPA in most natural oils. The fatty acids have usually been in the triglyceride or ethyl ester forms, and occasionally in the free acid and phospholipid forms. Docosapentaenoic acid (DPA n-3) is also a common component of such materials. The new understanding of possible mechanisms of action of EPA developed by the inventors has, however, led to the realisation that the purer the EPA is, the better the activity is likely to be. This is not just a question of dose, although that is indeed a valuable aspect of the application of pure EPA. From the point of view of a patient, particularly a mentally disturbed patient, it is obviously better to give, say, 1 g of EPA as a 95% pure preparation than, say, 5 g of a 19% pure preparation providing the same total amount of EPA. The patient is much more likely to comply with the lower volumes required with the highly purified compound.

The purification of EPA is difficult and complex. Because its five double bonds must all be in the right positions in the carbon chain and must all be in the cis configuration, EPA is difficult to synthesize. In nature EPA is almost always found mixed with other fatty acids in the forms of triglycerides and phospholipids. The principles of purification of EPA are well known to those skilled in the art and include low temperature crystallisation, urea fractionation, lithium crystallisation, fractional distillation, high pressure liquid chromatography, supercritical carbon dioxide chromatography and various other forms of chromatography using silica gels and other column packings. The application of these known techniques has been difficult to apply in practice on a large scale and only recently has pure EPA (more than 90% pure and preferably more than 95% pure) become available. In one version of the purification process, natural fish oil triglycerides rich in EPA are saponified and the fatty acids converted to the ethyl ester form. A preparation enriched in ethyl EPA is then prepared by molecular distillation with collection of the appropriate fraction. This fraction is then converted to a preparation containing over 80% of ethyl EPA by urea precipitation. The final preparation, of more than 96% pure ethyl EPA, is then achieved by either silica gel chromatography or high pressure liquid chromatography.

EPA can be synthesised but with great difficulty because of its thirty-two isomers, only one of which involves all the double bonds in the cis configuration and which is biologically active. It is usually therefore prepared from natural EPA-containing sources including micro algae and other micro-organisms, a wide range of different marine oils from fish, shellfish and marine mammals and, increasingly, from genetically modified micro-organisms or higher plants. EPA from any of these sources may be used in the invention. These provide sources of the acid, its derivatives and its metabolites.

The EPA may be used in the form of the natural oils or preferably in partially purified or fully purified extracts or semi-synthetic derivatives containing preferably more than 70% of the pure compound (the free acid and/or its derivatives) and very preferably more than 90% or more than 95% of the pure compound. Pure EPA-triglyceride or the pure ethyl ester of EPA are particularly suitable for these purposes. It is increasingly evident that EPA binds to highly specific sites in cells and that the binding can be interfered with by other fatty acids which can thus interfere with the activity of the EPA itself (D F Horrobin, Progr Drug Res, 2002). The best therapeutic results will therefore be obtained when the final pharmaceutical dosage form contains less than 10% in total and less than 3% individually of other fatty acids which might interfere with the action of EPA. Preferably the final dosage form should contain less than 5% in total and less than 2% individually of other fatty acids which might interfere with the action of EPA. The fatty acid of most concern in this context is the related fatty acid docosahexaenoic acid (DHA). Other fatty acids to be taken into consideration in this calculation are linoleic acid (LA), arachidonic acid (AA). Preferably, the EPA contains less than 10% in aggregate and less than 3% individually of DHA, LA, AA. Still preferably, the EPA contains less than 5% in aggregate and less than 2% individually of DHA, LA, AA. It may also be preferred that there is less than 2% AA in the EPA. EPA preparations of 1% or less DHA, LA or AA may be used. Alternatively, an EPA preparation in which DHA, LA or AA, is substantially absent may be employed.

The EPA may also be in the form of the free acid, a sodium, potassium, lithium or other salt, any ester, including an ethyl ester or a cholesterol ester, an amide, a phospholipid, or a tri-, di- or monoglyceride. The EPA may also be in the form of a 2-substituted derivative or other derivative which slows down its rate of oxidation but does not otherwise change its biological action on psychiatric or brain disorders to any substantial degree (N. Willumsen et al., Biochimica Biophysica Acta, 1998, 1369: 193-203). Other derivatives which are able to raise the levels of the fatty acid in the blood or tissues may be used. The preferred form of EPA is the ethyl ester or the triglyceride. These are particularly well tolerated by the gastrointestinal tract.

Because substantial amounts of EPA are always converted to DPA when administered to humans, it is likely that DPA will have effects similar to EPA. Similarly, other lipid mediators as metabolites of EPA or DPA may be used including series 3 prostoglandins and thromboxanes and series 5 leukotrienes.

The aspects of the present invention therefore include the treatments, methods and uses as described but where the EPA is replaced by DPA or a lipid mediator metabolite of EPA or DPA.

The EPA may be administered in any appropriate dosage form known to those skilled in the art. For oral administration, as examples, hard or soft gelatin or agar or other non-protein capsules, or any type of microcapsules are all appropriate, as are flavoured liquids and emulsions. The absence of smell with the pure EPA means that, unlike the situation with fish oils or less pure products, there is little risk of gastrointestinal upsets, or regurgitation of gas, or foul-smelling breath. For topical administration the EPA may be incorporated into any appropriate cream, ointment or emulsion. The pure EPA has no odour, which is a major advantage over fish oil and less purified products with regard to topical administration. For intravenous administration, the EPA, for example in the form of the ethyl ester, may be prepared in sterile vials and then mixed with any commercial intravenous lipid formulation for administration to the patient. Alternatively the EPA may be injected directly by slow intravenous injection or an intravenous sterile emulsion may be made for administration to the patient.

The preparation comprising EPA can be administered at a rate of 0.5 g/day, 1 g/day or 2 g/day. In cases where the initial rate of administration of EPA is 1 g/day or 2 g/day, the rate of administration can be reduced to 0.5 g/day, 2, 3, 4 or 5 weeks after the start of treatment with EPA.

The present invention further provides a method for treating severe melancholic depression, comprising administering, to a subject, a preparation comprising DPA.

The present invention still further provides a method for treating severe melancholic depression, comprising administering, to a subject, a preparation comprising a metabolite of EPA selected from series 3 prostoglandins and thromboxanes, and series 5 leukotrienes.

The present invention also provides the use of DPA, series 3 prostoglandins, series 3 thromboxanes, or series 5 leukotrienes in the manufacture of a medicament for the treatment of severe melancholic depression.

EPA is not only useful as a monotherapy in the treatment of severe melancholic depression. It can be co-administered with standard antidepressant drugs and can substantially enhance the response of patients suffering from severe melancholic depression to standard therapy, and also reduce many of the side effects of standard therapy.

The present invention further provides a method for treating severe melancholic depression, comprising co-administering a preparation comprising EPA with standard drugs which have antidepressant actions including tricyclic and related antidepressants, noradrenaline reuptake inhibitors, serotonin reuptake inhibitors, monoamine oxidase inhibitors and drugs with atypical antidepressant actions, either involving the same formulation or the same packaging.

Since its introduction in the sixties, the Hamilton Depression Rating Scale (HDRS) has become the established system for determining the severity of a subject's depression. It consists of questions (items) relating to 17 symptoms associated with depression which can be answered by a subject (FIG. 3). The overall score of a subject provides a guide to the severity of the subject's depression. It has also been found that scoring highly in specific areas of the test can provide an indication of the type of depression a patient is suffering from.

When dealing with subjects suffering from severe melancholic depression it is important to ensure that the correct medication is prescribed as early as possible. Severely melancholic subjects are often reluctant to take, and maintain taking, medication so it is important that, having persuaded then to start a program, positive results are achieved as early as possible to ensure compliance.

In addition, the present invention provides a method for identifying patients susceptible to therapeutic benefit from treatment with EPA comprising: testing the subject using the Hamilton Depression Rating Scale (HDRS); selecting subjects scoring a maximum of 2 on at least one of the items of the HDRS selected from early awakening (item 6), appetite loss (item 12) and weight loss (item 16).

The following results show, by way of example, the unexpected, positive, effect of EPA on severely melancholically depressed patients.

### BRIEF DESCRIPTION OF THE FIGURES

FIG. 1: shows the derivation of EPA from α-linolenic acid by a series of three reactions.

FIG. 2: shows the main types of highly unsaturated fatty acids.

FIG. 3: shows an example of the Hamilton Depression Rating Scale.

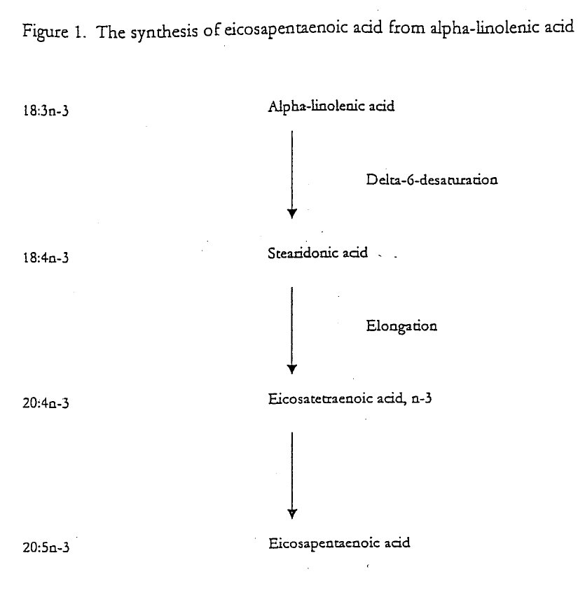

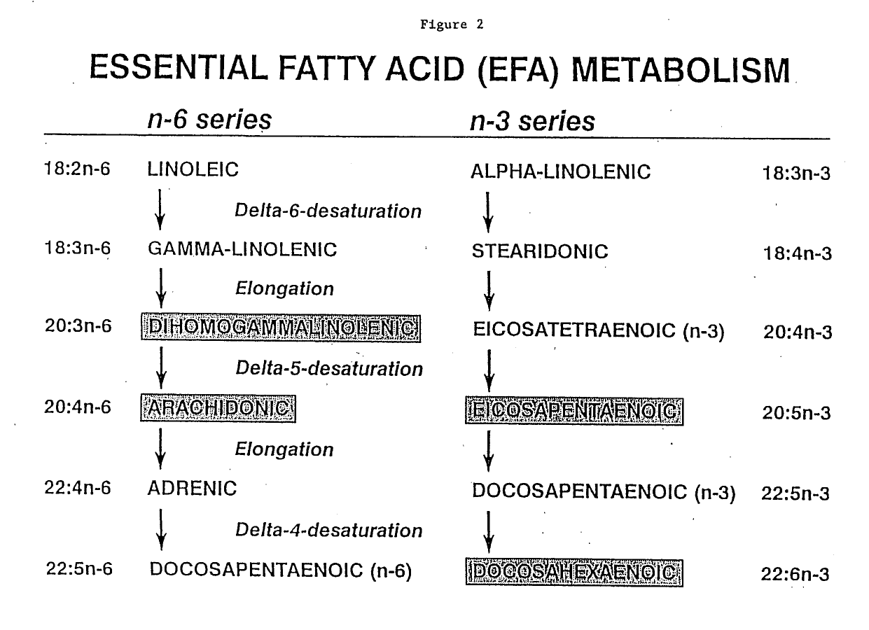

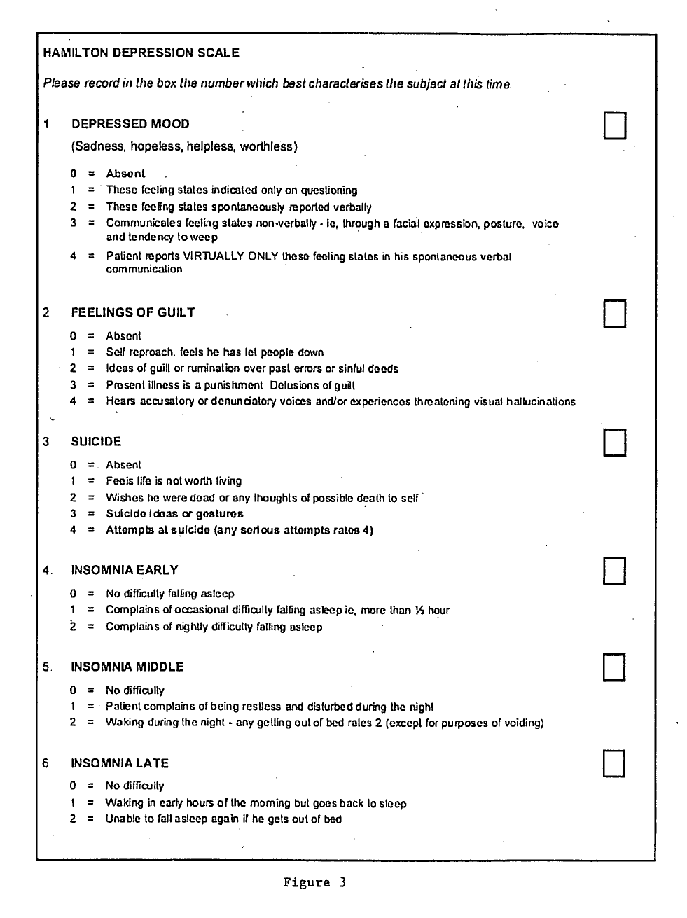

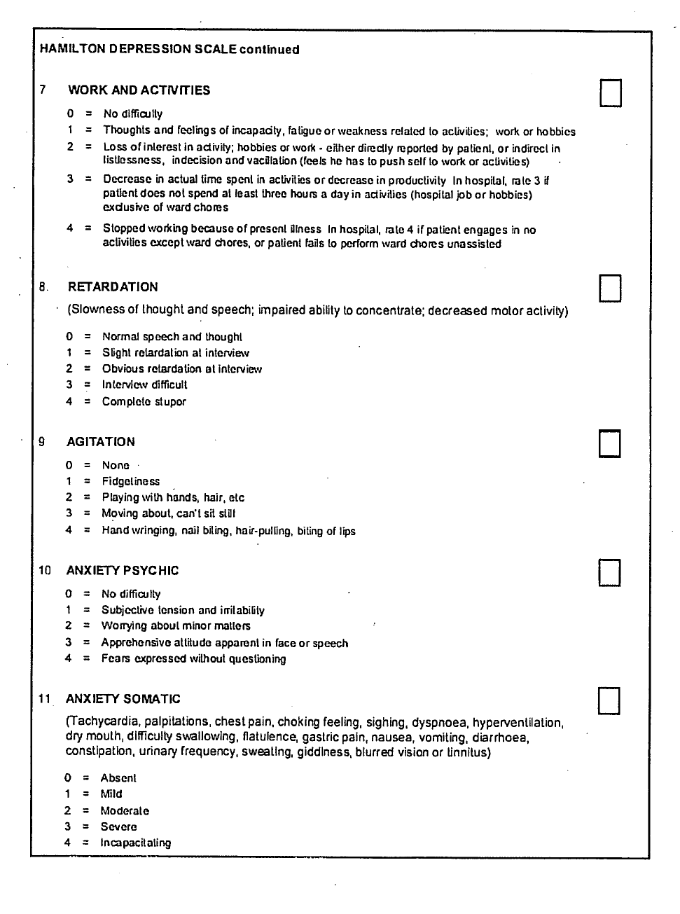

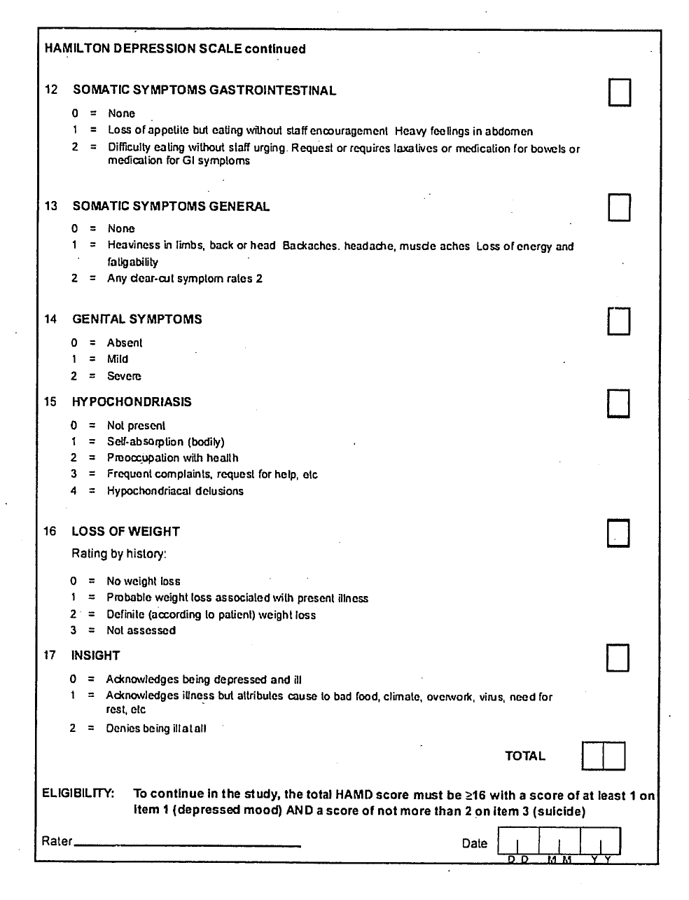

### EXAMPLES

### Example 1

### Treatment of Depressed Patients with Melancholic Features with Pure EPA

A study was performed on outpatients with a new episode of depression using 96% pure EPA, as the ethyl ester, as a monotherapy for treatment of depression.

The primary objective was to examine the effects of 96% pure EPA versus placebo on the Hamilton Depression Rating Scale (HDRS) in patients with a new or recurrent episode of depression.
1. The Bech Scale (sum of 6 items as specified) was used as the instrument to measure efficacy across all subjects, i.e. the reduction of the Bech Score in the course of the trial, by comparing this change in the active groups with that in the placebo group. The Bech Scale consists of 6 items from the original HDRS: depression, guilt, work/interest, psychomotor retardation, anxiety psychic and general somatic symptoms (loss of energy, tiredness) and has been successfully used in several trials (Bech 2002 Acta Psychiatry Scand vol 106; p 252-264).
2. For the differentiation of the subgroups of subjects, item 6 (early morning awakening), item 12 (loss of appetite) and item 16 (weight loss) were chosen from the HDRS, to identify subjects suffering from severe melancholic features. These items are also related to the definition of the melancholia specifier according the DSM-IV (American Psychiatric Association: 4^{th} Edition, 2000). This specification was only done once, i.e. at the baseline visit (v1). Patients with a score of 2 in any of these 3 items were regarded as having severe melancholic items and therefore belonging the severe melancholic group, the others were regarded as not belonging to this group. The characteristics could change in the course of treatment, therefore it was important that only the condition at baseline was used for the definition.
   Results:

The Bech score of the unsplit population is shown in Table 1 and of the split population in Table 2.

In the ITT group (Intention to Treat group, incorporating all subjects) (Table 1) the placebo group changed by 3.9 points, whereas the active group changed by 4.0 points, i.e. no significant difference could be demonstrated.

By splitting the groups according to the presence or absence of severe melancholic signs, we found in the non-severe melancholic group a change of 5.0 with placebo and of 4.1 with 96% pure EPA, which was not significantly different. However, surprisingly we found that in the patients with severe melancholic signs, the placebo led to a change of only 2.9 points, whereas 96% pure EPA improved by 3.8 points (p<0.05).

TABLE 1
ITT Group

| | placebo | | | EPA (any dose) | | |
|---|---|---|---|---|---|---|
| | | | Std | | | Std. |
| | Count | Mean | Deviation | Count | Mean | Deviation |
| Bech v1 | 20 | 10.7 | 1.3 | 57 | 10.3 | 2.0 |
| Bech l2 | 20 | 7.5 | 3.6 | 57 | 8.1 | 3.0 |
| Bech l4 | 20 | 6.7 | 3.7 | 57 | 6.6 | 3.5 |
| Bech l6 | 20 | 6.8 | 5.0 | 57 | 6.3 | 3.8 |

Bech v1 = Baseline week 1,
Bech l2: after 2 weeks;
Bech l4: after 4 weeks and
Bech l6: after 6 weeks (l = using last observation carried forward)

TABLE 2
Bech - Split into Severe Melancholic subgroups

| | | placebo | | | EPA (any dose) | | |
|---|---|---|---|---|---|---|---|
| | | Count | Mean | Std Deviation | Count | Mean | Std Deviation |
| No severe | Bech v1 (baseline) | 9 | 10.8 | 1.5 | 29 | 10.2 | 1.9 |
| melancholic | Bech l2 (week 2) | 9 | 6.2 | 4.6 | 29 | 8.8 | 2.8 |
| signs | Bech l4 (week 4) | 9 | 5.7 | 4.2 | 29 | 6.3 | 4.2 |
| | Bech l6 (week 6) | 9 | 5.8 | 4.4 | 29 | 6.1 | 4.3 |
| With severe | Bech v1 (baseline) | 11 | 10.5 | 1.1 | 28 | 10.3 | 2.1 |
| melancholic | Bech l2 (week 2) | 11 | 8.5 | 2.2 | 28 | 7.3 | 3.0 |
| signs | Bech l4 (week 4) | 11 | 7.5 | 3.3 | 28 | 7.0 | 2.8 |
| | Bech l6 (week 6) | 11 | 7.6 | 5.4 | 28 | 6.5 | 3.4 |

An analysis of a less frequently used, but pre-specified sub score, i.e. the depression sub score, which is the sum of item 1 (depression), item 2 (guilt) and item 3 (suicidality), confirmed the findings. The use of a statistical comparative test (ANVOVA analysis) of the outcome, with the baseline as covariate (as prespecified), led to a significant superiority of 96% pure EPA in the patients with severe melancholic signs (Table 3 and 4).

TABLE 3
Depression subscore (item1, item2, item3) - ITT

| | Placebo | | | EPA (any dose) | | |
|---|---|---|---|---|---|---|
| | | | Std | | | Std |
| | Count | Mean | Deviation | Count | Mean | Deviation |
| Dep (baseline) | 20 | 4.1 | .9 | 57 | 4.3 | 1.2 |
| Dep (outcome, | 20 | 2.9 | 2.6 | 57 | 2.3 | 1.8 |
| LOCF) | | | | | | |

TABLE 4
Depression subscore - Split into Severe Melancholic subgroups

| | | Placebo | | | EPA (any dose) | | |
|---|---|---|---|---|---|---|---|
| | | Count | Mean | Std Deviation | Count | Mean | Std Deviation |
| No severe | dep (baseline) | 9 | 4.0 | .9 | 29 | 4.4 | 1.3 |
| melancholic signs | dep (outcome) | 9 | 2.3 | 2.0 | 29 | 2.4 | 1.9 |
| With severe | dep (baseline) | 11 | 4.3 | .9 | 28 | 4.1 | 1.1 |
| melancholic signs | dep (outcome) | 11 | 3.6 | 2.9 | 28 | 2.3 | 1.8 |

The patients with the severe melancholic signs showed an improvement of only 0.7 points in the placebo group, but of 1.8 in the active group. This was statistically significant (p<0.05).

It will be appreciated that in clinical trials, a p-value of <0.05 is considered clinically significant, whereas a p-value >0.05 is not.

These data surprisingly show that 96% pure EPA treats the core symptoms of depression in patients with severe melancholic signs, a group of patients, which tends to show non-response to standard treatment.

### Example 2

### A Study Relating to the Use of Pure EPA as Add On Therapy

Interestingly, the defined split into the severe melancholic groups also defined responsive subgroups when analysing the results of earlier trials, where 96% pure EPA was used as an add on to standard therapy in non-responsive patients. The following presents the Bech-Score over time in the severe melancholic groups, contrasted with those subjects showing no severe melancholic signs.

TABLE 1
Bech score, for visits 1 (baseline) to visit 5 (outcome, 12 weeks)

| | | Treatment Group | | | | | |
|---|---|---|---|---|---|---|---|
| | | placebo | | | EPA (any dose) | | |
| | | Count | Mean | Std Deviation | Count | Mean | Std Deviation |
| No severe | Bech v1 (baseline) | 12 | 9.8 | 1.2 | 30 | 9.8 | 1.9 |
| melancholic | Bech l2 (week 2) | 12 | 8.6 | 1.9 | 30 | 8.3 | 2.9 |
| signs | Bech l4 (week 4) | 12 | 7.5 | 3.1 | 30 | 7.3 | 2.6 |
| | Bech l8 (week 8) | 12 | 7.2 | 3.0 | 30 | 6.7 | 2.7 |
| | Bech l12 (week 12) | 12 | 7.0 | 3.4 | 30 | 6.7 | 3.3 |
| With severe | Bech v1 (baseline) | 5 | 10.0 | 1.2 | 22 | 9.0 | 2.1 |
| melancholic | Bech l2 (week 2) | 5 | 9.2 | .4 | 22 | 8.1 | 2.2 |
| signs | Bech l4 (week 4) | 5 | 7.6 | .9 | 22 | 6.8 | 2.0 |
| | Bech l8 (week 8) | 5 | 7.2 | 1.3 | 22 | 5.9 | 2.6 |
| | Bech l12 (week 12 | 5 | 7.2 | 2.9 | 22 | 5.3 | 2.2 |

Despite the relatively small number of patients, especially in the placebo group of patients with severe melancholic signs, the outcome (baseline as covariate) at 12 weeks, using an ANVOVA model with the last observation carried forward, was statistically superior in the 96% pure EPA group, compared to placebo.

Again, in patients with severe melancholic features, 96% pure EPA shows a significant superiority over the placebo. The improvement in the placebo group was 2.8 points but in the groups showing severe melancholic signs, 3.7 points was observed. This was statistically significant (p<0.05).

The clinical data retrieved from both of these tests showed the surprising effect of EPA on patients suffering from severe melancholic depression. There was a marked and unexpected improvement in severely melancholic patients treated with EPA over those prescribed the placebo treatment.

Thus, by using the three-item HDRS test to initially screen for subjects suffering from severe melancholic depression, it is possible to identify those subjects who are most likely to benefit from treatment with EPA and the surprising effects shown by the present invention to be attributable thereto.

1. A method for treating severe melancholic depression, comprising administering to a subject a preparation comprising EPA.

2. A method according to claim 1, wherein the preparation comprises a composition comprising 90% pure EPA, or metabolite(s) thereof.

3. A method according to claim 1, wherein the preparation comprises a composition comprising 95% pure EPA, or metabolite(s) thereof.

4. A method according to claim 1, wherein the preparation is administered at a rate of 0.5 g/day.

5. A method according to claim 1, wherein the preparation is administered at a rate of 1 g/day.

6. A method according to claim 1, wherein the preparation is administered at a rate of 2 g/day.

7. A method according to claim 5, wherein, after 2 weeks from the start of treatment, the rate of administration is reduced to 0.5 g/day.

8. A method for identifying patients suffering from depression susceptible to therapeutic benefit from treatment with EPA, comprising testing the subject using the Hamilton Depression Rating Scale (HDRS); selecting subjects scoring a maximum of 2 on at least one of the items of the HDRS selected from; early awakening (item 6), appetite loss (item 12) and weight loss (item 16)

9. A method for treating severe melancholic depression, comprising co-administering a preparation comprising EPA with an antidepressant drug.

10. A method according to claim 9 wherein the EPA and antidepressant drug are in the same formulation.

11. A method according to claim 9 wherein the EPA and antidepressant drug are in the separate formulations.

12. A method according to claim 9 wherein the antidepressant is selected from: tricyclic and related antidepressants; noradrenaline reuptake inhibitors; serotonin reuptake inhibitors; monoamine oxidase inhibitors; and drugs with atypical antidepressant actions.
